(19) United States
(12) Patent Application Publication
(10) Pub. No.: US 2022/0211317 A1
(43) Pub. Date: Jul. 7, 2022

(54) UPPER LIMB TRAINING SYSTEM AND METHOD AND READABLE STORAGE MEDIUM

(71) Applicant: Zhengzhou University, Zhengzhou (CN)

(72) Inventors: Yuxia Hu, Zhengzhou (CN); Tongda Shen, Zhengzhou (CN); Lipeng Zhang, Zhengzhou (CN); Shaoting Yan, Zhengzhou (CN)

(21) Appl. No.: 17/577,628

(22) Filed: Jan. 18, 2022

(30) Foreign Application Priority Data
Dec. 15, 2021 (CN) 202111535181.4

(51) Int. Cl. A61B 5/375; A61B 5/374; A61B 5/378; G09B 19/00

(52) U.S. Cl. CPC A61B 5/375; A61B 5/374; A61B 2505/09; G09B 19/003; A61B 5/378

(57) ABSTRACT

The present application relates to the field of rehabilitation robotics, and discloses an upper limb training system and method and a readable storage medium. The system includes a training guidance unit, a signal acquisition unit, a signal processing unit, and a training unit. The training guidance unit shows training content to a subject to guide the subject to perform motor imagery. The signal acquisition unit acquires an electroencephalography (EEG) signal of the subject and sends the EEG signal to the signal processing unit. The signal processing unit recognizes a steady-state visual evoked potential (SSVEP) and a movement-related cortical potential (MRCP) generated by the subject in response to the training content. When the SSVEP is detected, the training unit generates a training instruction to assist the subject in active training, thereby improving the accuracy of brain control.

### CROSS REFERENCE TO RELATED APPLICATIONS

This application claims priority to Chinese Patent Application No. 2021115351814, filed on Dec. 15, 2021, the entire contents of which are incorporated herein by reference.

### TECHNICAL FIELD

The present application relates to the field of rehabilitation robotics, and specifically to an upper limb training system and method and a readable storage medium.

### BACKGROUND

The brain-computer interface technology is used in rehabilitation training systems to allow patients to actively participate in a rehabilitation training process, utilizing the neuroplasticity mechanisms of brains to enhance a rehabilitation training effect. However, current brain-computer interface-based rehabilitation training systems basically provide repetitive mechanical training for patients' limbs according to established procedures, and it is difficult to conduct limb rehabilitation training according to the actual state of the patients, resulting in low control accuracy and poor training activeness of the patients, which affects the rehabilitation effect of the patients.

### SUMMARY

In view of this, embodiments of the present application provide an upper limb training system and method and a readable storage medium, to resolve the problem of existing rehabilitation training systems that it is difficult to conduct limb rehabilitation training according to the actual state of the patients, resulting in low control accuracy and poor training activeness of the patients.

According to a first aspect, embodiments of the present application provide an upper limb training system, including: a training guidance unit, configured to show training content to a subject; a signal acquisition unit, communicatively connected to the training guidance unit, and configured to acquire an electroencephalography (EEG) signal of the subject and send the EEG signal to a signal processing unit; the signal processing unit, communicatively connected to the signal acquisition unit, and configured to receive the EEG signal sent by the signal acquisition unit and recognize a steady-state visual evoked potential (SSVEP) and a movement-related cortical potential (MRCP) included in the EEG signal; and a training unit, communicatively connected to the signal processing unit, and configured to generate a training instruction when the SSVEP is recognized, determine a movement intention of the subject according to the MRCP, and assist the subject in active training according to the training instruction.

The upper limb training system provided in the embodiments of the present application includes a training guidance unit, a signal acquisition unit, a signal processing unit, and a training unit. The training guidance unit shows training content to a subject to guide the subject to perform motor imagery. The signal acquisition unit acquires an EEG signal of the subject and sends the EEG signal to the signal processing unit for signal processing, to recognize a SSVEP and a MRCP generated by the subject in response to the training content. When the SSVEP is detected, the training unit can generate a training instruction to assist the subject in active training, thereby improving the accuracy of brain control and mustering the activeness of participation of the subject. Moreover, a movement intention of the subject is recognized according to the MRCP, the activeness of participation of the subject can be further determined, and the training status of the subject is learned in time, thereby ensuring the training effect of the subject.

With reference to the first aspect, in a first embodiment of the first aspect, the signal acquisition unit includes: a plurality of first acquisition electrodes, configured to acquire a first EEG signal of a frequency band in which the MRCP is located; and a plurality of second acquisition electrodes, configured to acquire a second EEG signal of a frequency band in which the SSVEP is located.

The signal acquisition unit in the upper limb training system provided in the embodiments of the present application includes a plurality of first acquisition electrodes and a plurality of second acquisition electrodes. The first acquisition electrodes and the second acquisition electrodes are respectively used to acquire an EEG signal of the frequency band in which the MRCP is located and an EEG signal of the frequency band in which the SSVEP is located. In this way, EEG signals in different frequency bands are separately acquired, to avoid that mixed acquisition of EEG signals affects a training progress.

With reference to the first aspect, in a second embodiment of the first aspect, the training guidance unit includes: an action setting module, configured to set a training action of the subject; a display module, configured to show the training action blinking at a preset frequency; and a frequency setting module, configured to set a training quantity and a trial duration of the training action.

The training unit in the upper limb training system provided in the embodiments of the present application includes an action setting module, a display module, and a frequency setting module. The action setting module sets a training action of the subject, the frequency setting module sets a training quantity and a trial duration of the training action, and the display module shows the set training action blinking at a preset frequency to evoke the SSVEP of the subject, so that when observing the training content shown by the training guidance unit, the subject can perform corresponding motor imagery to generate a MRCP, thereby improving the accuracy of brain control and mustering the activeness of the subject to participate in upper limb training.

With reference to the first aspect, in a third embodiment of the first aspect, the upper limb training system further includes: a training evaluation unit, communicatively connected to the signal processing unit, and configured to evaluate a training completion rate of the training content by the subject according to a first recognition rate of the SSVEP and a second recognition rate of the MRCP.

The upper limb training system provided in the embodiments of the present application further includes a training evaluation unit. The training evaluation unit evaluates a training completion rate of the training content by the subject according to a first recognition rate of the SSVEP and a second recognition rate of the MRCP in the EEG signal, so that the participation activeness of the upper limb training of the subject is evaluated by combining the accuracy of brain control and the detection rate of a movement intention, thereby improving the upper limb training effect of the subject.

According to a second aspect, embodiments of the present application provide an upper limb training method, applied to the upper limb training system in the first aspect or any embodiment of the first aspect. The upper limb training method includes: obtaining training content corresponding to a subject; acquiring an EEG signal of the subject in response to the training content, and recognizing a SSVEP and a MRCP from the EEG signal; and triggering a training instruction based on the SSVEP, determining a movement intention of the subject based on the MRCP, and assisting the subject in active training according to the training instruction.

In the training method provided in the embodiments of the present application, training content corresponding to a subject is obtained, an EEG signal of the subject in response to the training content is acquired, a SSVEP and a MRCP are recognized from the EEG signal, a training instruction is triggered based on the SSVEP, a movement intention of the subject is determined based on the MRCP, and the subject in active training is assisted according to the training instruction. In this way, the SSVEP triggers the generation of the training instruction to assist the subject in active training, thereby improving the accuracy of brain control.

Moreover, the movement intention of the subject according to the MRCP is recognized, the activeness of the subject to participate in upper limb training can be mustered, and the training status of the subject is learned in time, thereby improving the training effect of the subject.

With reference to the second aspect, in a first embodiment of the second aspect, the recognizing a SSVEP from the EEG signal includes: acquiring a second EEG signal of the subject, where the second EEG signal is an EEG signal of a frequency band in which the SSVEP is located; filtering out an interference signal in the second EEG signal, and obtaining a target frequency of the SSVEP; and recognizing, from the second EEG signal with the interference signal filtered out, a SSVEP that meets the target frequency.

In the training method provided in the embodiments of the present application, an interference signal in the second EEG signal is filtered out, and a target frequency of the SSVEP is obtained; and a SSVEP that meets the target frequency is recognized from the second EEG signal with the interference signal filtered out. In this way, the SSVEP that is included in the second EEG signal and responds to the training content can be accurately determined.

With reference to the first embodiment of the second aspect, in a second embodiment of the second aspect, the obtaining a target frequency of the SSVEP includes: obtaining stimulation frequencies of reference signals corresponding to the SSVEP; calculating correlation between the SSVEP and the reference signal of each stimulation frequency; and determining a stimulation frequency of a reference signal with the largest correlation as the target frequency of the SSVEP.

In the training method provided in the embodiments of the present application, stimulation frequencies of reference signals corresponding to the SSVEP are obtained, correlation between the SSVEP and the reference signal of each stimulation frequency is calculated, and a stimulation frequency of a reference signal with the largest correlation is determined as the frequency of the SSVEP, so that it is ensured that the target frequency can accurately represent the evocation of the SSVEP of the subject by the training content, and the SSVEP included in the EEG signal can be accurately recognized.

With reference to the first embodiment of the second aspect, in a third embodiment of the second aspect, the acquiring a MRCP of the subject in response to the training content includes: acquiring a first EEG signal of the subject, where the first EEG signal is an EEG signal of a frequency band in which the MRCP is located; an interference signal in the first EEG signal is filtered out, and extracting feature information of the MRCP; and inputting the feature information into a preset classification model, and determining the MRCP in response to the training content, where the preset classification model is obtained through classified training according to training sample data and actions corresponding to the training sample data.

In the training method provided in the embodiments of the present application, a first EEG signal of the subject is acquired, an interference signal in the first EEG signal is filtered out, extracting feature information of the MRCP, inputting the feature information into a preset classification model, and the MRCP in response to the training content is determined, where the preset classification model is obtained through classified training according to training sample data and actions corresponding to the training sample data. The MRCP in the EEG signal is recognized to determine the movement intention of the subject in response to current training content. In this way, the activeness of the subject to participate in the training content can be determined, thereby improving the participation experience of the subject.

With reference to the second aspect, in a fourth embodiment of the second aspect, the method further includes: recognizing a first recognition rate of the SSVEP and a second recognition rate of the MRCP from the EEG signal; and calculating a training completion rate of the subject based on weights of the first recognition rate and the second recognition rate in a training process.

In the training method provided in the embodiments of the present application, a first recognition rate corresponding to the SSVEP and a second recognition rate corresponding to the MRCP are acquired, and a training completion rate of the subject is calculated according to weights of the first recognition rate and the second recognition rate in a training process. In this way, the SSVEP and the MRCP are combined to implement training evaluation of the subject, so that on the basis of implementing the combination of the accuracy of brain control and the detection rate of the movement intention, the evaluation of the participation activeness of upper limb training of the subject is implemented, thereby improving the training effect of the subject.

According to a third aspect, embodiments of the present application provide a computer-readable storage medium, storing a computer instruction, where the computer instruction is used for causing a computer to perform the upper limb training method in the second aspect or any embodiment of the second aspect.

### BRIEF DESCRIPTION OF THE DRAWINGS

To describe the technical solutions in specific embodiments of the present application or the prior art more clearly, the following briefly introduces the accompanying drawings required for describing the specific embodiments or the prior art. Apparently, the accompanying drawings in the following description show some embodiments of the present application, and a person of ordinary skill in the art may still derive other drawings from these accompanying drawings without creative efforts.

FIG. 1 is a structural block diagram of an upper limb training system according to an embodiment of the present application;

FIG. 2 is a schematic diagram of acquisition of an EEG signal according to an embodiment of the present application;

FIG. 3 is a schematic diagram of setting of a training guidance unit according to an embodiment of the present application;

FIG. 4 is another structural block diagram of an upper limb training system according to an embodiment of the present application;

FIG. 5 is a flowchart of an upper limb training method according to an embodiment of the present application;

FIG. 6 is another flowchart of an upper limb training method according to an embodiment of the present application;

FIG. 7 is another flowchart of an upper limb training method according to an embodiment of the present application; and

FIG. 8 is a schematic test diagram of an upper limb training method according to an embodiment of the present application.

### DETAILED DESCRIPTION OF EMBODIMENTS

In order to make the objectives, technical solutions, and advantages of the embodiments of the present application clearer, the technical solutions in the embodiments of the present application will be clearly and completely described below in conjunction with the accompanying drawings in the embodiments of the present application. Obviously, the described embodiments are some of the embodiments of the present application, rather than all of the embodiments. All other embodiments obtained by persons skilled in the art based on the embodiments of the present application without creative efforts fall within the protection scope of the present application.

Current brain-computer interface-based rehabilitation training systems basically provide repetitive mechanical training for patients' limbs according to established procedures, and it is difficult to conduct limb rehabilitation training according to the actual state of the patients, resulting in low control accuracy and poor training activeness of the patients, which affects the rehabilitation effect of the patients.

Based on this, the technical solution of the present application is as follows. A SSVEP of a subject is recognized to drive a training unit in an upper limb training system to complete a training action, so that the accuracy of action execution of the training unit in the upper limb training system is improved, the MRCP of the subject is recognized to evaluate a movement intention of the subject, and the activeness of the subject to participant in training can be evaluated, thereby ensuring the training effect of the subject.

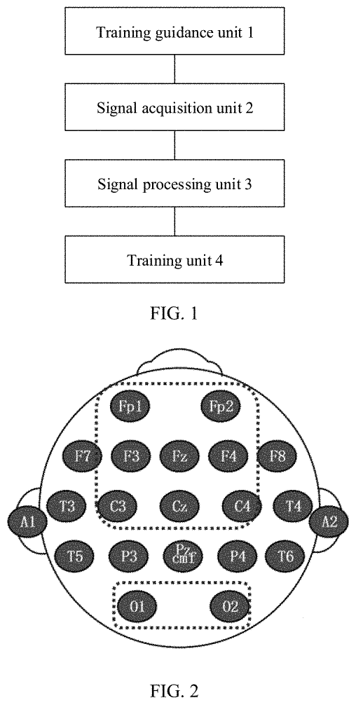

According to embodiments of the present application, an embodiment of an upper limb training system is provided. As shown in FIG. 1, the upper limb training system includes a training guidance unit 1, a signal acquisition unit 2, a signal processing unit 3, and a training unit 4. The training guidance unit 1 is communicatively connected to the signal acquisition unit 2. The signal processing unit 3 is communicatively connected to the signal acquisition unit 2. The training unit 4 is communicatively connected to the signal processing unit 3. Specifically, communication interfaces are disposed in the training guidance unit 1, the signal acquisition unit 2, the signal processing unit 3, and the training unit 4. The communication interfaces may be wired interfaces or wireless interfaces. The units implement a communication connection through the communication interfaces. The communication connection may be a wired connection, for example, a communication bus (an address bus, a data bus, a control bus or the like) connection. The communication connection may be alternatively a wireless connection, for example, a Bluetooth connection or a Wi-Fi connection.

A subject is an object with an upper limb movement disorder. Training content is a training operation to be performed by the subject. The training operation may be lifting an arm, lowering an arm, turning a wrist to the left, turning a wrist to the right, moving an arm to the left, moving an arm to the right, flexing and extending a finger or the like, or certainly may be another upper limb operation. This is not specifically limited herein. The training guidance unit 1 is configured to show the training content to the subject. The training content is shown to the subject in a picture guidance manner, so that the subject watches the training guidance unit and attempts to perform a corresponding action in a picture.

The signal acquisition unit 2 is communicatively connected to the training guidance unit 1. The signal acquisition unit 2 may acquire an EEG signal of the subject and send the EEG signal to a signal processing unit for corresponding processing. The signal acquisition unit 2 may be a wearable device. The wearable device includes a dry electrode configured to acquire an EEG signal. A particular acquisition frequency such as 300 Hz may be set during acquisition of an EEG signal. An EEG signal of the subject is acquired according to the 10-20 International Standard.

The signal processing unit 3 is communicatively connected to the signal acquisition unit 2. The signal processing unit 3 can receive the EEG signal sent by the signal acquisition unit 2 and recognize a SSVEP and a MRCP included in the EEG signal. The SSVEP is a potential evoked when the subject watches the training content shown by the training guidance unit 1.

The MRCP is used for representing a movement intention of the subject. The MRCP is generated when the subject watches the training content and attempts to perform corresponding motor imagery.

The training unit 4 is communicatively connected to the signal processing unit 3. The training unit 4 can generate a training instruction when the SSVEP is recognized, so that the training unit 4 is driven to assist the subject in completing the training operation corresponding to the training content. In addition, when assisting the subject to perform training, the training unit 4 can determine the movement intention of the subject according to the detected MRCP.

The upper limb training system provided in this embodiment includes a training guidance unit, a signal acquisition unit, a signal processing unit, and a training unit. The training guidance unit shows training content to a subject to guide the subject to perform motor imagery. The signal acquisition unit acquires an EEG signal of the subject and sends the EEG signal to the signal processing unit for signal processing, to recognize a SSVEP and a MRCP generated by the subject in response to the training content. When the SSVEP is detected, the training unit can generate a training instruction to assist the subject in active training, thereby improving the accuracy of brain control and mustering the activeness of participation of the subject. Moreover, a movement intention of the subject is recognized according to the MRCP, the activeness of participation of the subject can be further determined, and the training status of the subject is learned in time, thereby ensuring the training effect of the subject.

Optionally, the foregoing signal acquisition unit 2 may include a plurality of first acquisition electrodes and a plurality of second acquisition electrodes. The first acquisition electrodes are configured to acquire a first EEG signal of a frequency band in which the MRCP is located. The plurality of second acquisition electrodes are configured to acquire a second EEG signal of a frequency band in which the SSVEP is located.

Specifically, the frequency band in which the MRCP is located is 0.1 Hz to 1 Hz, and the frequency band in which the SSVEP is located is 5 Hz to 45 Hz. The first acquisition electrodes and the second acquisition electrodes may be dry electrodes. The first acquisition electrodes and the second acquisition electrodes are disposed according to the 10-20 International Standard System. As shown in FIG. 2, the first acquisition electrodes acquire an EEG signal of the frequency band in which the MRCP is located, and the second acquisition electrodes acquire an EEG signal of the frequency band in which the SSVEP is located. The signal acquisition unit 2 includes 24 acquisition channels, that is, 24 acquisition electrodes. 21 channels are used as synchronous channels for acquisition of an EEG signal, and 3 channels are used as expansion channels for acquisition of an EEG signal, and may be configured to acquire a myoelectric signal, an electrocardiogram signal, among other electrophysiological signals. During acquisition of an EEG signal, 21 channels are used to acquire an EEG signal at a frequency of 300 Hz. Channels in an area labeled with a dotted box in the upper part of FIG. 2 are used to recognize the MRCP, and the MRCP is used to represent the movement intention, including channels Fp1, Fp2, F3, F4, Fz, C3, C4, and Cz. That is, eight first acquisition electrodes respectively acquire first EEG signals corresponding to the channels Fp1, Fp2, F3, F4, Fz, C3, C4, and Cz. Channels in an area labeled with a dotted box in the lower part of FIG. 2 are used to recognize the SSVEP, including channels 01 and 02. That is, two second acquisition electrodes respectively acquire second EEG signals corresponding to the channels 01 and 02.

The signal acquisition unit in the upper limb training system provided in this embodiment includes a plurality of first acquisition electrodes and a plurality of second acquisition electrodes. The first acquisition electrodes and the second acquisition electrodes are respectively used to acquire an EEG signal of the frequency band in which the MRCP is located and an EEG signal of the frequency band in which the SSVEP is located. In this way, EEG signals in different frequency bands are separately acquired, to avoid that mixed acquisition of EEG signals affects a training progress.

Optionally, the foregoing training guidance unit 1 may include an action setting module, a display module, and a frequency setting module.

Specifically, the action setting module is configured to set a training action of the subject, for example, lifting an arm, lowering an arm, turning a wrist to the left, turning a wrist to the right, moving an arm to the left, moving an arm to the right or flexing and extending a finger. The training action may be selected according to an actual case of the subject, to improve the recognition rate of an EEG signal, and the action setting module may be used to set an action, so that the subject can perform repeated training on the action.

The display module is configured to show the training action blinking at a preset frequency. The preset frequency is a frequency at which the training action is blinked on the display module. The preset frequency is used for evoking the SSVEP of the subject. The display module may be a display screen. There may be a virtual arm on the display screen for showing the training action blinking at the preset frequency. During training, the subject may be requested to observe the blinking of the training action and try the best to perform a corresponding movement task.

The frequency setting module is configured to set a training quantity and a trial duration of the training action. The training quantity is a quantity of times of repeatedly training each training action. The trial duration is a time that a current training process lasts.

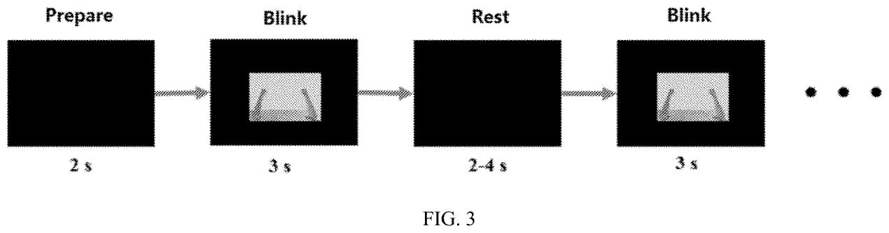

Lifting an arm is used as an example, as shown in FIG. 3. When training starts, the action setting module may be used to set the training action to lifting an arm, and at the same time a preparation time of 2 s may be further set. A training task interface corresponding to the display module is then entered. A picture of a virtual arm blinks at the center of the screen at a frequency of 10 Hz on the training task interface. A blinking duration (for example, 3 s) and the training quantity (for example, 5 times) may be set by the frequency setting module. In a training process, the subject is requested to focus on the blinking of the training action and try the best to perform a corresponding movement task. The black screen is entered after the blinking ends. The subject may rest 2 s to 4 s, and then a next training action is entered. A task quantity and the blinking duration are set to be consistent with those previously.

The training unit in the upper limb training system provided in this embodiment includes an action setting module, a display module, and a frequency setting module. The action setting module sets a training action of the subject, the frequency setting module sets a training quantity and a trial duration of the training action, and the display module shows the set training action blinking at a preset frequency to evoke the SSVEP of the subject, so that when observing the training content shown by the training guidance unit, the subject can perform corresponding motor imagery to generate a MRCP, thereby improving the accuracy of brain control and mustering the activeness of the subject to participate in upper limb training.

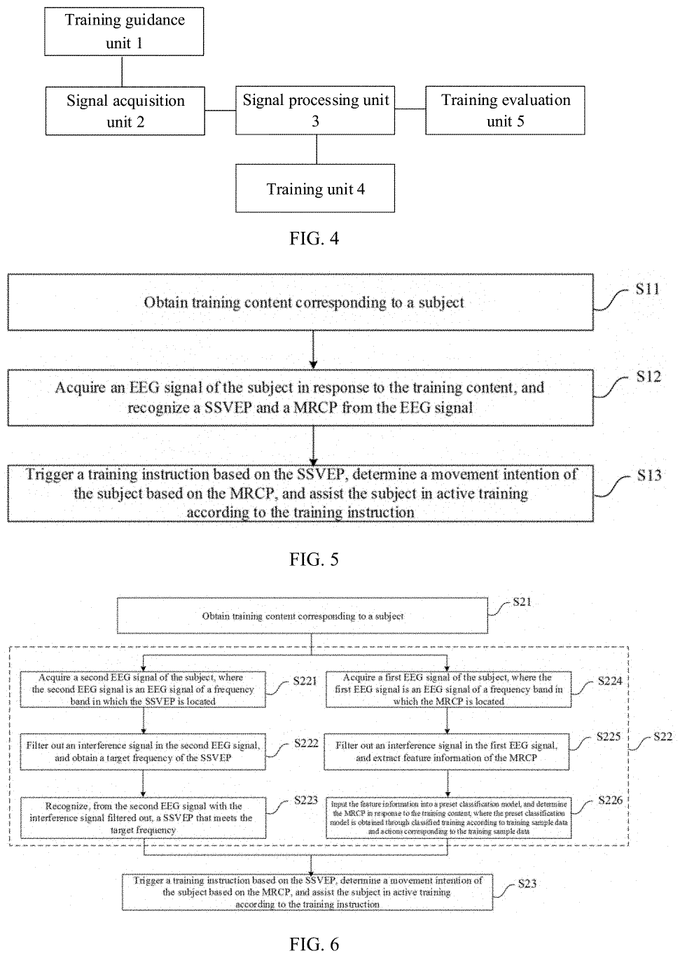

Optionally, as shown in FIG. 4, the foregoing upper limb training system further includes a training evaluation unit 5. The training evaluation unit 5 is communicatively connected to the signal processing unit 4. The training evaluation unit 5 may evaluate a corresponding training completion rate upon completion of the training content by the subject according to a first recognition rate of the SSVEP, a second recognition rate of the MRCP, and proportions of the SSVEP and the MRCP during training. The first recognition rate is a recognition rate of recognizing the SSVEP of the subject in response to the training action from the EEG signal. The second recognition rate is a recognition rate of recognizing the MRCP of the subject in response to the training action from the EEG signal.

The upper limb training system provided in this embodiment further includes a training evaluation unit. The training evaluation unit evaluates a training completion rate of the training content by the subject according to a first recognition rate of the SSVEP and a second recognition rate of the MRCP in the EEG signal, so that the participation activeness of the upper limb training of the subject is evaluated by combining the accuracy of brain control and the detection rate of a movement intention, thereby improving the upper limb training effect of the subject.

Embodiments of the present application provide an embodiment of an upper limb training method. It needs to be noted that the steps illustrated in the flowchart of the accompanying drawings may be performed in a computer system such as a set of computer-executable instructions, and that, although a logical sequence is shown in the flowchart, in some cases the steps shown or described may be performed in an order different from that shown herein.

This embodiment of the present application provides an upper limb training method, which may be applied to the foregoing upper limb training system. FIG. 5 is a flowchart of an upper limb training method according to an embodiment of the present application. As shown in FIG. 5, the procedure includes the following steps:

S11: Obtain training content corresponding to a subject.

The upper limb training system the training guidance unit is used to set the training content. Specifically, a user of the upper limb training system may use a keyboard or a mouse to select a training action, and set a training quantity of the training action. A frequency at which the training action is blinked in a picture form and a duration of the training action, and the like. After the setting is completed, the user may use a keyboard or a mouse to enter a complete instruction. In this case, the training guidance unit of the upper limb training system may generate corresponding training content in response to the complete instruction.

S12: Acquire an EEG signal of the subject in response to the training content, and recognize a SSVEP and a MRCP from the EEG signal.

The signal acquisition unit in the upper limb training system may acquire an EEG signal of the subject synchronously and in real time. When the subject responds to the training content, the signal acquisition unit may acquire an EEG signal of the subject in response to the training content and send the acquired EEG signal to the signal processing unit. The signal processing unit may filter out an interference signal in the EEG signal. A filter is used to filter the EEG signal to obtain an EEG signal of a frequency band in which the SSVEP is located and an EEG signal of a frequency band in which the MRCP is located, to further recognize the SSVEP corresponding to the training action from the EEG signal based on canonical correlation analysis (CCA), and the MRCP corresponding to the training action is recognized from the EEG signal based on features of the MRCP and a support vector machine (SVM).

S13: Trigger a training instruction based on the SSVEP, determine a movement intention of the subject based on the MRCP, and assist the subject in active training according to the training instruction.

The training instruction drives the training unit to assist the subject in completing an upper limb training action. When recognizing the SSVEP, the signal processing unit of the upper limb training system may transmit information to the training unit. Correspondingly, the training unit may generate the training instruction based on the SSVEP. In this case, the upper limb training system may use the signal acquisition unit to acquire the MRCP, to use the MRCP to determine whether the subject has a movement intention of attempting a training operation corresponding to the training content when the subject observes the training content shown by the training guidance unit, to further make the training unit be driven by the training instruction to assist the subject in performing a corresponding upper limb training.

In the upper limb training method provided in this embodiment, the SSVEP triggers the generation of the training instruction to assist the subject in active training, thereby improving the accuracy of brain control. Moreover, the movement intention of the subject according to the MRCP is recognized, the activeness of the subject to participate in upper limb training can be mustered, and the training status of the subject is learned in time, thereby improving the training effect of the subject.

This embodiment of the present application provides an upper limb training method, which may be applied to the foregoing upper limb training system. FIG. 6 is a flowchart of an upper limb training method according to an embodiment of the present application. As shown in FIG. 6. the procedure includes the following steps:

S21: Obtain training content corresponding to a subject. For detailed description, refer to the related description corresponding to step S11 in the foregoing embodiment. Details are not described again herein.

S22: Acquire an EEG signal of the subject in response to the training content, and recognize a SSVEP and a MRCP from the EEG signal.

Specifically, the foregoing step 202 may include the following substeps:

S221: Acquire a second EEG signal of the subject, where the second EEG signal is an EEG signal of a frequency band in which the SSVEP is located.

The frequency band in which the SSVEP is located is 5 Hz to 45 Hz. The upper limb training system may acquire the second EEG signal of the frequency band in which the SSVEP is located by using the signal acquisition unit.

S222: Filter out an interference signal in the second EEG signal, and obtain a target frequency of the SSVEP.

The target frequency is a stimulation frequency of the SSVEP corresponding to the training content. For example, the target frequency may be 7.5 Hz, or may be 8 Hz, or may be 10 Hz, or certainly may be another frequency value. This is not specifically limited herein. The upper limb training system may filter out the interference signal in the second EEG signal by using the signal processing unit. Because the frequency band in which the SSVEP is located is 5 Hz to 45 Hz. The signal processing unit may use a 5-Hz high pass filter to obtain an EEG signal with a frequency greater than 5 Hz by filtering the second EEG signal, and obtain a SSVEP corresponding to the training content from the EEG signal obtained through filtering.

Specifically, the foregoing step 222 may include the following sub-steps:

(1) Obtain stimulation frequencies of reference signals corresponding to the SSVEP.

The reference signal is a known signal for detecting the SSVEP. The stimulation frequency corresponding to the reference signal is an external stimulation frequency for guiding the subject to generate the reference signal. Optionally, the reference signal may be a sinusoidal signal. The stimulation frequency is a change frequency corresponding to the sinusoidal signal.

(2) Calculate correlation between the SSVEP and the reference signal of each stimulation frequency.

The signal processing unit in the upper limb training system calculates potential correlation between the SSVEP and the reference signal of each stimulation frequency according to CCA. Specifically, it is set that a multidimensional variable X is a SSVEP acquired by a plurality of channels of the signal acquisition unit, and a multidimensional variable Y represents the reference signal. Linear combinations between X and Y are x=X^{T}W_{X} and y=Y^{T}W_{Y}, to further find the maximum weight vectors W_{X} and W_{Y} of vectors x and y to calculate a correlation coefficient ρ. A calculation manner of the correlation coefficient is as follows:

$${max}_{{W}_{X} {W}_{Y}} ρ ( x , y ) = \frac{E [ {W}_{X}^{T} {XY}^{T} {W}_{Y} ]}{\sqrt{E [ {W}_{X}^{T} {XX}^{T} {W}_{X} ] E [ {W}_{Y}^{T} {YY}^{T} {W}_{Y} ]}} .$$

The target frequency of the SSVEP is detected in an unsupervised manner. Herein, the sinusoidal signal may be used as a reference signal Y. The reference signal Y is represented as follows:

$$Y = [ \begin{matrix}sin ( 2 π ft ) \\ cos ( 2 π ft ) \\ … \\ sin ( 2 π {N}_{h} ft ) \\ co s ( 2 π {N}_{h} ft )\end{matrix} ] ,$$

where f is the stimulation frequency of the reference signal, Nₕ is a quantity of harmonic waves, correlation between the SSEVP and the reference signal corresponding to each stimulation frequency is sequentially calculated.

(3) Determine a stimulation frequency of a reference signal with the largest correlation as the target frequency of the SSVEP.

The calculated correlation between the SSEVP and the reference signal corresponding to each stimulation frequency is compared to determine a stimulation frequency of a reference signal with the largest correlation. The stimulation frequency is determined as the target frequency of the SSEVP, that is, the frequency of the SSEVP in response to a training action.

S223: Recognize, from the second EEG signal with the interference signal filtered out, a SSVEP that meets the target frequency.

There may be SSVEPs of a plurality of frequencies in the second EEG signal with the interference signal filtered out. The signal processing unit may extract a SSVEP that meets the target frequency from the second EEG signal according to the target frequency of the SSVEP determined by the signal processing unit. The SSVEP that meets the target frequency is the SSVEP in response to the training content.

It needs to be noted that for different training actions, one same SSVEP frequency may be set, so that the SSVEP in response to the training content can be extracted more accurately from the EEG signal.

S224: Acquire a first EEG signal of the subject, where the first EEG signal is an EEG signal of a frequency band in which the MRCP is located.

The frequency band in which the MRCP is located is 0.1 Hz to 1 Hz. The upper limb training system may acquire the first EEG signal of the frequency band in which the MRCP is located by using the signal acquisition unit.

S225: Filter out an interference signal in the first EEG signal, and extract feature information of the MRCP.

Because the EEG signal is easily susceptible to interference signals such as electrooculography artifacts, myoelectric artifacts, and baseline drift. The signal processing unit may remove the interference signals such as electrooculography artifacts based on wavelet analysis, to obtain an EEG signal with a frequency between 0.1 Hz and 1 Hz according to a 0.1 to 1 Hz band pass filter.

The feature information is a feature corresponding to the training action. After obtaining the EEG signal with a frequency between 0.1 Hz and 1 Hz, the signal processing unit down samples the EEG signal, to reduce the sampling rate of the EEG signal to 10 Hz, and then after the training guidance unit shows a picture of the training action and the picture blinks for 1.5 s, EEG signal data in a prefrontal area is acquired as a feature signal corresponding to the training action. Each acquisition channel has 15 sampling points, and 15*8=120 feature signals may be obtained from eight acquisition channels. Frequency, amplitude, and other information included in the feature signals are feature information.

S226: Input the feature information into a preset classification model, and determine the MRCP in response to the training content, where the preset classification model is obtained through classified training according to training sample data and actions corresponding to the training sample data.

The preset classification model is obtained by training an SVM according to training sample data and actions corresponding to the training sample data. The preset classification model is configured to recognize a training action corresponding to a currently acquired MRCP. Specifically, during training of the preset classification model, a group of training data is first recorded. The training data includes 50 training actions, then feature data corresponding to the training actions is extracted, an SVM is then trained using the feature data corresponding to the actions and the training actions in the training data, and the trained classification model is configured to perform a class test on a MRCP. A linear kernel function is selected for a parameter of the SVM.

S23: Trigger a training instruction based on the SSVEP, determine a movement intention of the subject based on the MRCP, and assist the subject in active training according to the training instruction. For detailed description, refer to the related description corresponding to step S11 in the foregoing embodiment. Details are not described again herein.

In the upper limb training method provided in this embodiment, an interference signal in the second EEG signal is filtered out, a target frequency of the SSVEP is obtained, and a SSVEP that meets the target frequency is recognized from the second EEG signal with the interference signal filtered out. In this way, the SSVEP that is included in the second EEG signal and responds to the training content can be accurately determined. Further, obtaining stimulation frequencies of reference signals corresponding to the SSVEP, correlation between the SSVEP and the reference signal of each stimulation frequency is calculated, and determining a stimulation frequency of a reference signal with the largest correlation as the frequency of the SSVEP, so that it is ensured that the target frequency can accurately represent the evocation of the SSVEP of the subject by the training content, and the SSVEP included in the EEG signal can be accurately recognized. The MRCP in the EEG signal is recognized to determine the movement intention of the subject in response to current training content. In this way, the activeness of the subject to participate in the training content can be determined, thereby improving the participation experience of the subject.

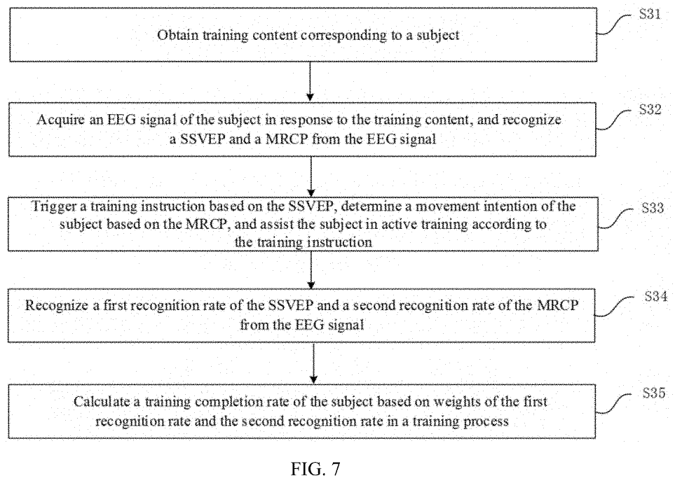

This embodiment provides an upper limb training method, which may be applied to the foregoing upper limb training system. FIG. 7 is a flowchart of an upper limb training method according to an embodiment of the present application. As shown in FIG. 7, the procedure includes the following steps:

S31: Obtain training content corresponding to a subject. For detailed description, refer to the related description corresponding to step S11 in the foregoing embodiment. Details are not described again herein.

S32: Acquire an EEG signal of the subject in response to the training content, and recognize a SSVEP and a MRCP from the EEG signal. For detailed description, refer to the related description corresponding to step S12 in the foregoing embodiment. Details are not described again herein.

S33: Trigger a training instruction based on the SSVEP, determine a movement intention of the subject based on the MRCP, and assist the subject in active training according to the training instruction. For detailed description, refer to the related description corresponding to step S11 in the foregoing embodiment. Details are not described again herein.

S34: Recognize a first recognition rate of the SSVEP and a second recognition rate of the MRCP from the EEG signal.

The first recognition rate is a recognition rate of the SSVEP in response to the training content. The second recognition rate is a recognition rate of the MRCP in response to the training content. Specifically, the first recognition rate and the second recognition rate may be obtained from training results of a plurality of times of upper limb training, for example, the subject is trained three times. The first recognition rate in the first time of training is 100%. A second recognition rate in the first time of training is 100%. A third recognition rate in the first time of training is 100%. The eventual first recognition rate is 99.3%. Similarly, a calculation manner of the second recognition rate is the same.

S35: Calculate a training completion rate of the subject based on weights of the first recognition rate and the second recognition rate in a training process.

Weights of the first recognition rate and the second recognition rate in a training process are used for representing levels of importance of training effects of the first recognition rate and the second recognition rate for the subject. The training completion rate is SOC=K1*ACC_{SSVEP}+K2*ACC_{M}. K1 is a weight of the first recognition rate, K2 is a weight of the second recognition rate, ACC_{SSVEP} is the first recognition rate, and ACC_{M} is the second recognition rate. Specifically, the weight of the first recognition rate may be the same as the weight of the second recognition rate. That is, K1=K2=0.5, SOC=(ACC_{SSVEP}+ACC_{M}).

In the upper limb training method provided in this embodiment, a first recognition rate corresponding to the SSVEP and a second recognition rate corresponding to the MRCP are acquired, and a training completion rate of the subject is calculated according to weights of the first recognition rate and the second recognition rate in a training process. In this way, the SSVEP and the MRCP are combined to implement training evaluation of the subject, so that on the basis of implementing the combination of the accuracy of brain control and the detection rate of the movement intention, the evaluation of the participation activeness of upper limb training of the subject is implemented, thereby improving the training effect of the subject.

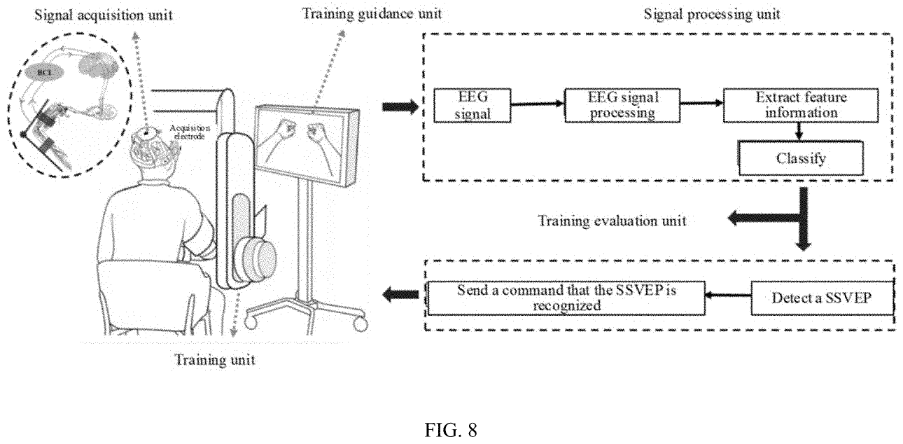

A specific example of an experiment is used for describing the upper limb training method in the foregoing embodiments. In the experiment, a total of 12 healthy subjects aged between 18 and 30 are chosen, including 8 men and 4 women, all being right-handed and having no nervous system diseases. The experimental environment is a room with normal light and adequate sound insulation. As shown in FIG. 8, a subject sits facing the training guidance unit in the upper limb training system, to avoid artifact signals generated from myoelectricity. The subject is required to relax the whole body and avoid unnecessary swallowing.

All 12 subjects are tested offline before the experiment. EEG signals of the subjects are acquired. An SVM classification model for recognizing a movement intention is calculated. A specific test process is as follows: A subject first wears the signal acquisition unit (for example, an EEG cap) and sits at a display module (for example, a display screen) corresponding to the training guidance unit, and training content is set by using an action setting module and a frequency setting module. Upper limb training starts after the setting of the training content is completed. After the training starts, the display screen turns dark. The subject has a preparation time of 2 s. A blinking image block of a training action appears on the display screen. The signal acquisition unit acquires and recognizes an EEG signal three seconds after the blinking image block starts blinking. For the SSVEP, 5 Hz to 45 Hz filtering is first performed on the EEG signal, and then, after a target frequency of the SSVEP is recognized using a CCA algorithm, an instruction is sent to the training unit (for example, a rehabilitation training robot). The training unit responds to the instruction and generates a corresponding training instruction, to perform corresponding training content. For a MRCP-based movement intention recognition method, an EEG signal 1.5 seconds after the blinking image block has blinked is intercepted to perform interference signal filtering and 0.1 Hz to 1 Hz filtering, and then feature information is extracted for classification and recognition. The display screen turns dark to continue with a next training action. This process is repeated to complete all training actions. Every subject needs to carry out 50 commands in each round of experiment, and three rounds of experiment are performed on each person.

In addition, a movement intention recognition method with mu-beta rhythms is used as a control group. An EEG signal 1.5 seconds after the blinking image block has blinked is intercepted to perform 8 Hz to 30 Hz filtering, and then spatial filtering is performed by using a two-class CSP algorithm. Energy of each channel after the spatial filtering is extracted as feature information. Classification and recognition are performed using an SVM. The display screen turns dark to continue with a next training action. This process is repeated to complete all training actions. Every subject needs to carry out 50 commands in each round of experiment, and three rounds of experiment are performed on each person.

Online test results of 12 subjects are shown in Tables 1, 2, and 3. Table 1 shows accuracy rates of performing training actions of a SSVEP-based training unit. The 12 subjects reach an average control accuracy rate of 99.3%. Table 2 shows accuracy rates of MRCP-based movement intention detection. The 12 subjects reach an average recognition rate of 82.7%. Table 3 shows a movement intention recognition method based on mu-beta rhythms. The 12 subjects reach an average detection accuracy rate of 77.2%. However, three subjects have relatively poor recognition results. The three subjects may be subjects with motor imagery illiteracy. Compared with the movement intention detection method based on mu-beta rhythms, the recognition results of a MRCP-based movement intention detection method are more stable, and there is no subject with motor imagery illiteracy. Table 4 shows evaluation results of completion rates of active training based on a SSVEP and a MRCP. The 12 subjects reach an average training completion rate of 0.91.

Table 1 shows accuracy rates of performing training actions of the SSVEP-based training unit.

| | | | | Average |
|---|---|---|---|---|
| | | | | accuracy |
| Subject | First time | Second time | Third time | rate |
| LB | 100.0% | 100.0% | 100.0% | 100.0% |
| WP | 100.0% | 100.0% | 100.0% | 100.0% |
| LXP | 98.0% | 100.0% | 98.0% | 98.7% |
| YYF | 100.0% | 98.0% | 100.0% | 99.3% |
| WYW | 98.0% | 100.0% | 100.0% | 99.3% |
| LJJ | 100.0% | 98.0% | 100.0% | 99.3% |
| TGD | 98.0% | 98.0% | 100.0% | 98.7% |
| WJT | 100.0% | 98.0% | 100.0% | 99.3% |
| HSM | 100.0% | 98.0% | 100.0% | 99.3% |
| ZLP | 98.0% | 100.0% | 98.0% | 98.7% |
| LRQ | 98.0% | 100.0% | 100.0% | 99.3% |
| YDR | 100.0% | 100.0% | 98.0% | 99.3% |
| Average | | | | 99.3% |
| accuracy | | | | |
| rate | | | | |

Table 2 shows accuracy rates of MRCP-based movement intention detection of subjects.

| | | | | Average |
|---|---|---|---|---|
| | | | | accuracy |
| Subject | First time | Second time | Third time | rate |
| LB | 76.0% | 94.0% | 88.0% | 86.0% |
| WP | 72.0% | 70.0% | 92.0% | 78.0% |
| LXP | 90.0% | 76.0% | 88.0% | 84.7% |
| YYF | 88.0% | 80.0% | 86.0% | 84.7% |
| WYW | 88.0% | 90.0% | 80.0% | 86.0% |
| LJJ | 74.0% | 74.0% | 82.0% | 76.7% |
| TGD | 92.0% | 70.0% | 82.0% | 81.3% |
| WJT | 82.0% | 94.0% | 84.0% | 86.7% |
| HSM | 84.0% | 80.0% | 88.0% | 84.0% |
| ZLP | 86.0% | 72.0% | 82.0% | 80.0% |
| LRQ | 86.0% | 90.0% | 76.0% | 84.0% |
| YDR | 70.0% | 92.0% | 80.0% | 80.7% |
| Average | | | | 82.7% |
| accuracy | | | | |
| rate | | | | |

Table 3 shows accuracy rates of mu-beta rhythm-based movement intention detection of subjects.

| | | | | Average |
|---|---|---|---|---|
| | | | | accuracy |
| Subject | First time | Second time | Third time | rate |
| LB | 58.0% | 62.0% | 68.0% | 62.7% |
| WP | 84.0% | 78.0% | 86.0% | 82.7% |
| LXP | 92.0% | 80.0% | 84.0% | 85.3% |
| YYF | 58.0% | 60.0% | 56.0% | 58.0% |
| WYW | 86.0% | 92.0% | 80.0% | 86.0% |
| LJJ | 84.0% | 78.0% | 86.0% | 82.7% |
| TGD | 82.0% | 80.0% | 82.0% | 81.3% |
| WJT | 78.0% | 88.0% | 80.0% | 82.0% |
| HSM | 74.0% | 82.0% | 78.0% | 78.0% |
| ZLP | 88.0% | 78.0% | 86.0% | 84.0% |
| LRQ | 52.0% | 60.0% | 56.0% | 56.0% |
| YDR | 84.0% | 88.0% | 90.0% | 87.3% |
| Average | | | | 77.2% |
| accuracy | | | | |
| rate | | | | |

Table 4 shows evaluation results of training completion of the active and passive upper limb rehabilitation training systems based on the recognition accuracy of SSVEPs and MRCPs.

| | | | | Average |
|---|---|---|---|---|
| | | | | completion |
| Subject | First time | Second time | Third time | rate |
| LB | 0.88 | 0.97 | 0.94 | 0.93 |
| WP | 0.86 | 0.85 | 0.96 | 0.89 |
| LXP | 0.94 | 0.88 | 0.93 | 0.917 |
| YYF | 0.94 | 0.89 | 0.93 | 0.92 |
| WYW | 0.93 | 0.95 | 0.90 | 0.927 |
| LJJ | 0.87 | 0.86 | 0.91 | 0.88 |
| TGD | 0.95 | 0.84 | 0.91 | 0.90 |
| WJT | 0.91 | 0.96 | 0.92 | 0.93 |
| HSM | 0.92 | 0.89 | 0.94 | 0.917 |
| ZLP | 0.92 | 0.86 | 0.90 | 0.893 |
| LRQ | 0.92 | 0.95 | 0.88 | 0.917 |
| YDR | 0.85 | 0.96 | 0.89 | 0.90 |
| Average | | | | 0.91 |
| completion | | | | |
| rate | | | | |

In the upper limb raining method in is embodiment, the recognition accuracy of the SSVEPs and the MRCPs are combined to implement active training evaluation of the subject, so that on the basis of implementing the combination of the accuracy of brain control and the detection rate of the movement intention, the evaluation of the participation activeness of upper limb training of the subject is implemented, thereby improving the training effect of the subject.

Embodiments of the present application further provide a non-transitory computer storage medium, storing a computer-executable instruction. The computer-executable instruction can perform a processing method in the upper limb training method in any foregoing method embodiment. The storage medium may be a magnetic disk, an optical disc, a read-only memory (ROM), a random access memory (RAM), a flash memory, a hard disk drive (HDD), a solid-state drive (SSD) or the like. The storage medium may also include a combination of the above-mentioned types of memories.

Although the embodiments of the present application are described in conjunction with the accompanying drawings, various modifications and variations may be made by those skilled in the art without departing from the spirit and scope of the present application, and such modifications and variations fall within the scope defined by the appended claims.

1. An upper limb training system, comprising:
a training guidance unit, configured to show training content to a subject;
a signal acquisition unit, communicatively connected to the training guidance unit, and configured to acquire an electroencephalography (EEG) signal of the subject and send the EEG signal to a signal processing unit;
the signal processing unit, communicatively connected to the signal acquisition unit, and configured to receive the EEG signal sent by the signal acquisition unit and recognize a steady-state visual evoked potential (SSVEP) and a movement-related cortical potential (MRCP) comprised in the EEG signal; and
a training unit, communicatively connected to the signal processing unit, and configured to generate a training instruction when the SSVEP is recognized, determine a movement intention of the subject according to the MRCP, and assist the subject in active training according to the training instruction.

2. The upper limb training system according to claim 1, wherein the signal acquisition unit comprises:
a plurality of first acquisition electrodes, configured to acquire a first EEG signal of a frequency band in which the MRCP is located; and
a plurality of second acquisition electrodes, configured to acquire a second EEG signal of a frequency band in which the SSVEP is located.

3. The upper limb training system according to claim 1, wherein the training guidance unit comprises:
an action setting module, configured to set a training action of the subject;
a display module, configured to show the training action blinking at a preset frequency; and
a frequency setting module, configured to set a training quantity and a trial duration of the training action.

4. The upper limb training system according to claim 1, further comprising:
a training evaluation unit, communicatively connected to the signal processing unit, and configured to evaluate a training completion rate of the training content by the subject according to a first recognition rate of the SSVEP and a second recognition rate of the MRCP.

5. An upper limb training method, applicable to the upper limb training system according to claim 1, and comprising:
obtaining training content corresponding to a subject;
acquiring an electroencephalography (EEG) signal of the subject in response to the training content, and recognizing a steady-state visual evoked potential (SSVEP) and a movement-related cortical potential (MRCP) from the EEG signal; and
triggering a training instruction based on the SSVEP, determining a movement intention of the subject based on the MRCP, and assisting the subject in active training according to the training instruction.

6. The method according to claim 5, wherein the recognizing a SSVEP from the EEG signal comprises:
acquiring a second EEG signal of the subject, wherein the second EEG signal is an EEG signal of a frequency band in which the SSVEP is located;
filtering out an interference signal in the second EEG signal, and obtaining a target frequency of the SSVEP; and
recognizing, from the second EEG signal with the interference signal filtered out, a SSVEP that meets the target frequency.

7. The method according to claim 6, wherein the obtaining a target frequency of the SSVEP comprises:
obtaining stimulation frequencies of reference signals corresponding to the SSVEP;
calculating correlation between the SSVEP and the reference signal of each stimulation frequency; and
determining a stimulation frequency of a reference signal with the largest correlation as the target frequency of the SSVEP.

8. The method according to claim 6, wherein the acquiring a MRCP of the subject in response to the training content comprises:
acquiring a first EEG signal of the subject, wherein the first EEG signal is an EEG signal of a frequency band in which the MRCP is located;
filtering out an interference signal in the first EEG signal, and extracting feature information of the MRCP; and
inputting the feature information into a preset classification model, and determining the MRCP in response to the training content,
wherein the preset classification model is obtained through classified training according to training sample data and actions corresponding to the training sample data.

9. The method according to claim 5, further comprising:
recognizing a first recognition rate of the SSVEP and a second recognition rate of the MRCP from the EEG signal; and
calculating a training completion rate of the subject based on weights of the first recognition rate and the second recognition rate in a training process.

10. A computer-readable storage medium, storing a computer instruction, wherein the computer instruction is used for causing a computer to perform the upper limb training method comprising:
obtaining training content corresponding to a subject;
acquiring an electroencephalography (EEG) signal of the subject in response to the training content, and recognizing a steady-state visual evoked potential (SSVEP) and a movement-related cortical potential (MRCP) from the EEG signal; and
triggering a training instruction based on the SSVEP, determining a movement intention of the subject based on the MRCP, and assisting the subject in active training according to the training instruction.

11. The computer-readable storage medium according to claim 10, storing a computer instruction, wherein the computer instruction is used for causing a computer to perform the upper limb training method, wherein the recognizing a SSVEP from the EEG signal comprises:
acquiring a second EEG signal of the subject, wherein the second EEG signal is an EEG signal of a frequency band in which the SSVEP is located;
filtering out an interference signal in the second EEG signal, and obtaining a target frequency of the SSVEP; and
recognizing, from the second EEG signal with the interference signal filtered out, a SSVEP that meets the target frequency.

12. The computer-readable storage medium according to claim 11, storing a computer instruction, wherein the computer instruction is used for causing a computer to perform the upper limb training method, wherein the obtaining a target frequency of the SSVEP comprises:
obtaining stimulation frequencies of reference signals corresponding to the SSVEP;
calculating correlation between the SSVEP and the reference signal of each stimulation frequency; and
determining a stimulation frequency of a reference signal with the largest correlation as the target frequency of the SSVEP.

13. The computer-readable storage medium according to claim 11, storing a computer instruction, wherein the computer instruction is used for causing a computer to perform the upper limb training method, wherein the acquiring a MRCP of the subject in response to the training content comprises:
acquiring a first EEG signal of the subject, wherein the first EEG signal is an EEG signal of a frequency band in which the MRCP is located;
filtering out an interference signal in the first EEG signal, and extracting feature information of the MRCP; and
inputting the feature information into a preset classification model, and determining the MRCP in response to the training content,
wherein the preset classification model is obtained through classified training according to training sample data and actions corresponding to the training sample data.

14. The computer-readable storage medium according to claim 10, storing a computer instruction, wherein the computer instruction is used for causing a computer to perform the upper limb training method, further comprising:
recognizing a first recognition rate of the SSVEP and a second recognition rate of the MRCP from the EEG signal; and
calculating a training completion rate of the subject based on weights of the first recognition rate and the second recognition rate in a training process.
